(19) United States
(12) Patent Application Publication
(10) Pub. No.: US 2021/0166817 A1
(43) Pub. Date: Jun. 3, 2021

(54) INFORMATION PROCESSING APPARATUS AND NON-TRANSITORY COMPUTER READABLE MEDIUM

(71) Applicant: FUJI XEROX CO., LTD., Tokyo (JP)

(72) Inventors: Kengo TOKUCHI, Kanagawa (JP); Masahiro SATO, Kanagawa (JP)

(73) Assignee: FUJI XEROX CO., LTD., Tokyo (JP)

(21) Appl. No.: 16/856,505

(22) Filed: Apr. 23, 2020

(30) Foreign Application Priority Data
Dec. 3, 2019 (JP) 2019-218602

(51) Int. Cl. G16H 50/30; G16H 10/60; G16H 40/67; G06Q 20/40; A61B 5/16; A61B 5/0476

(52) U.S. Cl. CPC G16H 50/30; G16H 10/60; A61B 5/0476; G06Q 20/40; A61B 5/165; G16H 40/67

(57) ABSTRACT

An information processing apparatus includes a processor. The processor is configured to obtain biological information indicating a state of a subject user which is found when an instruction is provided from the subject user and to determine whether to restrict accepting of the instruction in accordance with the state indicated by the biological information.

### CROSS-REFERENCE TO RELATED APPLICATIONS

This application is based on and claims priority under 35 USC 119 from Japanese Patent Application No. 2019-218602 filed Dec. 3, 2019.

### BACKGROUND

### (i) Technical Field

The present disclosure relates to an information processing apparatus and a non-transitory computer readable medium.

### (ii) Related Art

Japanese Unexamined Patent Application Publication No. 2005-293209 discloses a personal authentication device which detects that an authorized user is forced to perform an illegal operation, as well as verifying the identification of a user by using existing biometric authentication.

Japanese Unexamined Patent Application Publication No. 2014-018234 discloses a health care management system that manages health care of a user by identifying the mental state and the physical state of the user.

Japanese Unexamined Patent Application Publication No. 2005-148982 discloses the following authentication method. The ID of a service user is obtained. Biological information concerning the service user is obtained. The obtained biological information is checked against registered biological information associated with the obtained ID so as to judge whether the service user is an authorized user.

### SUMMARY

Sometimes, people are unable to make a reasonable decision or judgement due to its physical or mental condition, for example.

Aspects of non-limiting embodiments of the present disclosure relate to preventing the execution of an instruction provided from a user depending on the state of the user.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided an information processing apparatus including a processor. The processor is configured to obtain biological information indicating a state of a subject user which is found when an instruction is provided from the subject user and to determine whether to restrict accepting of the instruction in accordance with the state indicated by the biological information.

### BRIEF DESCRIPTION OF THE DRAWINGS

An exemplary embodiment of the present disclosure will be described in detail based on the following figures, wherein:

FIG. 1 is a block diagram illustrating an example of the configuration of an information processing system according to the exemplary embodiment;

FIG. 2 is a block diagram illustrating an example of the configuration of an information processing apparatus according to the exemplary embodiment;

FIG. 3 illustrates a management table;

FIG. 4 illustrates another management table;

FIG. 5 illustrates a screen; and

FIG. 6 schematically illustrates users and robots.

### DETAILED DESCRIPTION

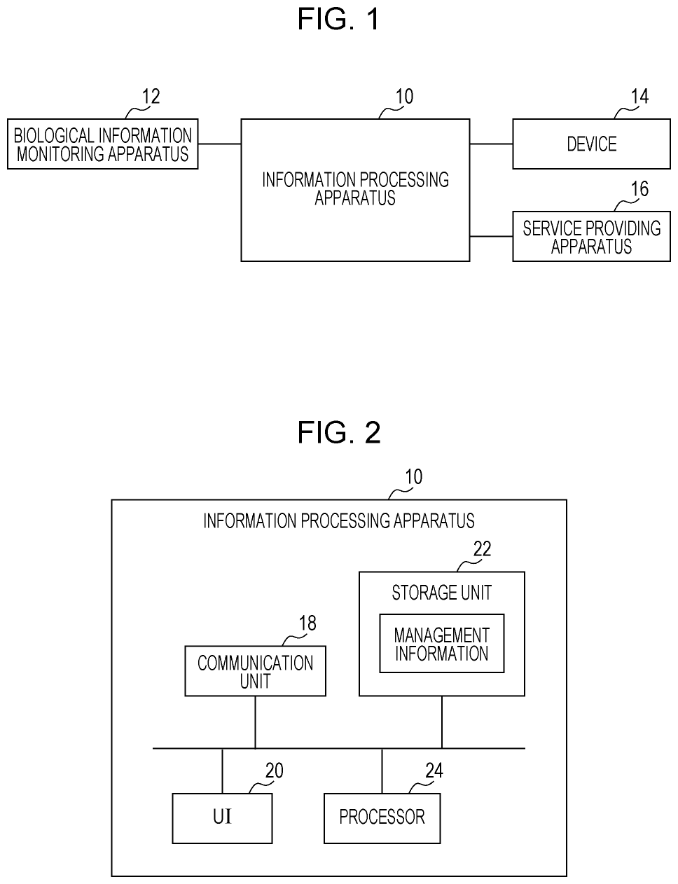

An information processing system according to the exemplary embodiment will be described below with reference to FIG. 1. FIG. 1 illustrates an example of the configuration of the information processing system according to the exemplary embodiment.

The information processing system according to the exemplary embodiment includes an information processing apparatus 10, one or plural biological information monitoring apparatuses 12, one or plural devices 14, and one or plural service providing apparatuses 16. Only the single information processing apparatus 10, biological information monitoring apparatus 12, device 14, and service providing apparatus 16 are shown in FIG. 1, but this configuration is only an example. The information processing system may include an apparatus and/or a device (such as a server) other than the information processing apparatus 10, the biological information monitoring apparatus 12, the device 14, and the service providing apparatus 16 shown in FIG. 1. The provision of the device 14 and the service providing apparatus 16 in the information processing system may be omitted.

Each apparatus or device included in the information processing system is configured to communicate with another apparatus or device included in the information processing system via a wired medium using a cable or a wireless medium. That is, each apparatus or device may be physically connected to another apparatus or device via a cable and send and receive information with each other. Alternatively, each apparatus or device may send and receive information with another apparatus or device by wireless communication. Examples of wireless communication are short-range wireless communication and Wi-Fi (registered trademark). Another wireless communication standard may be employed. Examples of short-range wireless communication are Bluetooth (registered trademark), radio frequency identifier (RFID), and near field communication (NFC). Each apparatus or device may alternatively communicate with another apparatus or device via a communication channel, such as a local area network (LAN) or the Internet.

Examples of the information processing apparatus 10 are a personal computer (hereinafter called a PC), a tablet PC, a smartphone, a cellular phone, and other apparatuses, such as a server. The information processing apparatus 10 may be a portable terminal device (such as a tablet PC, a smartphone, or a cellular phone), or a device placed on a desk or a table. For example, the information processing apparatus 10 may be a smart speaker having a communication function and including a microphone and a speaker. The information processing apparatus 10 may be installed indoors (on the floor or the ceiling of a room or on the table in a room) or outdoors. The information processing apparatus 10 may be a movable device, such as a self-running device.

The biological information monitoring apparatus 12 is configured to monitor biological information concerning a user. For example, the biological information monitoring apparatus 12 includes various sensors and electrodes, for example, and monitors biological information concerning a user. If multiple biological information monitoring apparatuses 12 are used, they may individually monitor different types of biological information. Alternatively, some or all of the biological information monitoring apparatuses 12 may monitor the same type of biological information. Conversely, the single biological information monitoring apparatus 12 may monitor one type of biological information or multiple types of biological information.

The biological information monitoring apparatus 12 sends monitored biological information to the information processing apparatus 10. Every time the biological information monitoring apparatus 12 monitors biological information, it may send the monitored information to the information processing apparatus 10. The biological information monitoring apparatus 12 may alternatively store monitored biological information and send it to the information processing apparatus 10 at predetermined regular intervals or at a timing given by a user. The biological information monitoring apparatus 12 may receive biological information monitored by another biological information monitoring apparatus 12 and send it together with biological information monitored by the biological information monitoring apparatus 12 to the information processing apparatus 10.

The biological information monitoring apparatus 12 may analyze biological information monitored by the biological information monitoring apparatus 12 or another biological information monitoring apparatus 12 and send information indicating the analysis results to the information processing apparatus 10. The biological information monitoring apparatus 12 may include a processor and a storage unit, and the processor may analyze biological information. Biological information may alternatively be analyzed by the information processing apparatus 10, the device 14, the service providing apparatus 16, or another apparatus.

The biological information monitoring apparatus 12 may include batteries and be driven by power supplied from them. The biological information monitoring apparatus 12 may alternatively be driven by receiving power from another apparatus, such as the information processing apparatus 10.

The biological information monitoring apparatus 12 may be attached to a user. For example, the biological information monitoring apparatus 12 may be a wearable device that is worn by a user and monitors biological information concerning the user. Specific examples of the biological information monitoring apparatus 12 are a device fixed on the user□s head (the forehead, for example), a bearable device attached to the user□s ear/ears (such as earphone/earphones or headphones), a device fixed to the user□s arm, hand, wrist, or finger (for example, a watch-type device, such as a smart watch), a device put on the user□s neck, a device fixed to the user□s torso (the abdomen or chest, for example), and a device fixed to the lower limb (the thigh, lower leg, knee, foot, or ankle, for example). The biological information monitoring apparatus 12 may be health equipment fixed to the user□s arm, hand, torso, or lower limb. The biological information monitoring apparatus 12 may be attached to another part of the body other than the above-described parts. The biological information monitoring apparatus 12 may be attached to each of multiple parts of the body.

Biological information is physiological information and anatomical information concerning various physiological and anatomical aspects of human bodies. The concept of biological information covers information concerning the brain activities (such as brain waves, brain blood flow, and brain magnetic field signal), the pulse rate, myoelectric information, such as myoelectric waveforms, the saliva (such as the amount of saliva), the pulse waves, the blood pressure, the blood flow, the pulse, the heart rate, the electrocardiogram waveforms, the eye movement, the body temperature, the amount of perspiration, gaze, voice, and the motion of a human body. Information obtained by a biomarker may be used as biological information. The above-described items of information are only examples of the biological information, and another item of physiological information or anatomical information may be used as the biological information. The biological information monitoring apparatus 12 may monitor one or multiple items of the above-described items of biological information.

The concept of biological information covers bioelectric potential information indicating the potentials generated from the body. The concept of bioelectric potential information covers brain waves obtained by measuring minute electric currents generated by the brain activities, electrocardiograms created by measuring minute electric currents generated by the heart pulsating beats, electromyograms created by measuring minute electric currents generated by the muscle activities, and skin potentials obtained by measuring minute electric currents generated in the skin. The above-described items of information are only examples of bioelectric potential information, and another item of bioelectric potential information may be used.

After receiving biological information from the biological information monitoring apparatus 12, the information processing apparatus 10 analyzes, stores, and outputs the biological information, and also stores and outputs information indicating the analysis results of the biological information. Analyzing of biological information may alternatively be conducted by the biological information monitoring apparatus 12, the device 14, the service providing apparatus 16, or another apparatus. To output biological information is to display it or output it as sound information, for example. To output information indicating the analysis results of biological information is to display it or output it as sound information, for example. The information processing apparatus 10 may send biological information and information indicating the analysis results to another apparatus.

The information processing apparatus 10 may contain one or plural biological information monitoring apparatuses 12. That is, one or plural biological information monitoring apparatuses 12 may be integrated into the information processing apparatus 10 so as to form a single apparatus. The entirety of the information processing apparatus 10 integrating one or plural biological information monitoring apparatuses 12 may be worn by a user to monitor biological information. That is, the information processing apparatus 10 may be a wearable device. Specific examples of the information processing apparatus 10 are a device fixed on the user□s head (the forehead, for example), a bearable device attached to the user□s ear/ears (such as earphone/earphones or headphones), a device fixed to the user□s arm, hand, wrist, or finger (for example, a watch-type device, such as a smart watch), a device put on the user□s neck, a device fixed to the user□s torso (the abdomen or chest, for example), and a device fixed to the lower limb (the thigh, lower leg, knee, foot, or ankle, for example). The information processing apparatus 10 may be health equipment fixed to the user□s hand, torso, or lower limb. The information processing apparatus 10 may be attached to another part of the body other than the above-described parts.

The information processing apparatus 10 and the biological information monitoring apparatus 12 may be separate apparatuses. For example, the information processing apparatus 10 may be a smart speaker, while the biological information monitoring apparatus 12 may be a wearable device worn by a user.

Examples of the device 14 are a PC, a tablet PC, a smartphone, a cellular phone, a robot (such as a humanoid robot, an animal robot, a robotic vacuum cleaner, and another type of robot), a projector, a display, such as a liquid crystal display, a recorder, a playback device, an image capturing device, such as a camera, a refrigerator, a rice steamer, a microwave oven, a coffee machine, a vacuum cleaner, a washing machine, an air conditioner, lighting equipment, health equipment, a watch, a clock, a surveillance camera, an automobile, a motorbike, a motorcycle, a bicycle, an aircraft (an unmanned aerial vehicle, such as a drone, for example), a game machine, a gas stove, an electronic bidet, a ventilation fan, a doorbell, an entrance monitoring system, an elevator, a door, a window, and various types of sensing devices (such as a temperature sensor, a humidity sensor, a voltage sensor, and a current sensor). The concept of the device 14 may cover almost all types of equipment. For example, information equipment, video equipment, and audio equipment may be examples of the device 14 in the exemplary embodiment.

The device 14 includes a communication unit, which is a communication interface, a storage unit that stores data, and a processor that controls the operation of the device 14. The device 14 may also include a user interface. The device 14 may send device identification information for identifying the device 14 to the information processing apparatus 10. The device identification information is the ID, name, model type, or address (such as media access control (MAC) address or Internet protocol (IP) address) of the device 14, for example.

The service providing apparatus 16 is configured to provide various services. Services are web services, for examples. Web services are services provided via a network, for example, the Internet, such as application providing services, web mail services, social networking services (SNS), services for providing video images and music, services for selling products, services for making reservations, search services, information providing services, and services for providing payment options. Other services may be provided by the service providing apparatus 16.

The service providing apparatus 16 includes a communication unit, which is a communication interface, a storage unit that stores data, and a processor that controls the operation of the service providing apparatus 16. The service providing apparatus 16 may also include a user interface. For example, upon receiving a request to provide a service, the processor of the service providing apparatus 16 provides a service in response to the request.

The configuration of the information processing apparatus 10 will be described below in detail with reference to FIG. 2. FIG. 2 illustrates an example of the configuration of the information processing apparatus 10.

The information processing apparatus 10 includes a communication unit 18, a user interface (UI) 20, a storage unit 22, and a processor 24. The information processing apparatus 10 may include another element other than the above-described elements.

The communication unit 18, which is a communication interface, has a function of sending data to another apparatus and a function of receiving data from another apparatus. The communication unit 18 may have a wireless communication function or a wired communication function. The communication unit 18 may communicate with another apparatus by using short-range wireless communication or via a communication channel, such as a LAN or the Internet. For example, the communication unit 18 receives biological information sent from the biological information monitoring apparatus 12. The communication unit 18 may send control information for controlling the operation of the biological information monitoring apparatus 12 to the biological information monitoring apparatus 12. The communication unit 18 may send control information for controlling the operation of the device 14 to the device 14 and receive information sent from the device 14. The communication unit 18 may send information indicating a request to provide a web service to the service providing apparatus 16 and receive information concerning a web service sent from the service providing apparatus 16.

The UI 20 includes at least one of a display and an operation unit. The display is a liquid crystal display or an electroluminescence (EL) display, for example. The operation unit is a keyboard, input keys, or an operation panel, for example. The UI 20 may be a touchscreen which serves both as the display and the operation unit. The UI 20 may include a microphone and/or a speaker which emits sound.

The storage unit 22 is a device forming one or multiple storage regions for storing data. The storage unit 22 is a hard disk drive, various memory devices (such as a random access memory (RAM), a dynamic random access memory (DRAM), and a read only memory (ROM)), another storage unit (such as an optical disc), or a combination thereof, for example. One or plural storage units 22 are included in the information processing apparatus 10.

In the storage unit 22, management information is stored. Management information is information for determining the state of a user, based on monitored biological information concerning the user. For example, reference biological information, state information indicating the state of a user, and instruction acceptability information are linked with each other and are registered in the management information.

The reference biological information is biological information that is assumed to be generated from a user being in the state indicated by the state information linked with this reference biological information. The reference biological information is determined by executing statistical processing, for example.

The state of a user is the mental state of the user, for example. It can be said that the reference biological information linked with the state information indicating the mental state of a user is biological information indicating the mental state of this user. The state of a user may be a state other than the mental state, such as the emotional state or the physical state. The state information concerning a user is information indicating whether the user is capable of making a reasonable decision or judgement when providing a certain instruction.

The instruction acceptability information is information indicating the acceptability of an instruction from a user, that is, whether to restrict the accepting of an instruction from a user. A determination as to whether to restrict the accepting of an instruction from a user is made based on the state of the user. Hereinafter, restricting the accepting of an instruction from a user may simply be called restricting an instruction from a user.

When a user is in no condition to make a reasonable decision or judgement, an instruction from this user is restricted. For example, if the state information concerning a user indicates that the user is in no condition to make a reasonable decision or judgement, the instruction acceptability information linked with this state information indicates that instructions from the user are restricted.

When a user is capable of making a reasonable decision or judgement, instructions from this user are not restricted. For example, if the state information concerning a user indicates that the user is capable of making a reasonable decision or judgement, the instruction acceptability information linked with this state information indicates that instructions from the user are not restricted.

Examples of an instruction from a user are an instruction to make a payment, an instruction to form a contract, and any other instructions involving a certain decision or judgement. An instruction from a user may be an instruction provided for the device 14 or the service providing apparatus 16. An instruction for the device 14 is an instruction for operating the device 14, for example. An instruction for operating the device 14 may include the content of operation to be performed on the device 14. An instruction for the service providing apparatus 16 is an instruction for using a service provided by the service providing apparatus 16, for example. Specific examples of an instruction for the service providing apparatus 16 are an instruction to make a payment for using a service provided by the service providing apparatus 16, an instruction to purchase a product, an instruction to purchase a service, an instruction to perform a login operation or conduct authentication for using a service provided by the service providing apparatus 16, an instruction to register for membership, and an instruction to cancel membership. An instruction from a user is not restricted to these instructions and may be any instruction involving a certain decision or judgement.

A user may provide an instruction to the information processing apparatus 10 by operating the UI 20, via sound, or by using biological information.

An instruction from a user may be input into the device 14 or the service providing apparatus 16 directly or via the information processing apparatus 10.

An instruction from a user may be input into a terminal device, such as a PC, a laptop PC, a smartphone, or a cellular phone, used by the user.

For each user, the reference biological information, the state information, and the instruction acceptability information may be linked with each other and be registered in the management information.

The processor 24 is configured to obtain biological information concerning a user which is found when an instruction is provided from the user and to determine whether to restrict the accepting of the instruction in accordance with the state indicated by the biological information.

When biological information concerning a user is monitored by the biological information monitoring apparatus 12, it is sent from the biological information monitoring apparatus 12 to the information processing apparatus 10, and the processor 24 obtains this biological information. The processor 24 may continuously obtain biological information concerning a user even if an instruction is not provided from the user, or obtain biological information monitored at a time when a user has provided an instruction. The processor 24 may alternatively obtain biological information monitored at a time when a user has provided an instruction and during a period prior to and subsequent to this time. The processor 24 may alternatively obtain biological information monitored during a period prior to a time when a user has provided an instruction or during a period subsequent to this time. Each of the period prior to and subsequent to a time when a user has provided an instruction, the period prior to this time, and the period subsequent to this time is a predetermined period, and may be changed, for example, by a user.

The processor 24 obtains biological information concerning a user which is monitored when an instruction is provided from the user from the biological information monitoring apparatus 12, and determines whether to restrict the accepting of the instruction in accordance with the state indicated by the biological information.

This will be explained more specifically. The processor 24 checks monitored biological information concerning a user against each item of reference biological information registered in the management information, and searches for an item of reference biological information which deviates from the monitored biological information by an amount within a permissible range. The processor 24 then identifies the state information linked with this item of reference biological information, and determines the state indicated by the state information to be the state of the user. The processor 24 determines the acceptability for the instruction from the user, that is, determines whether to restrict the instruction, in accordance with the instruction acceptability information linked with the state information. That is, the processor 24 determines based on the biological information concerning the user whether the state of the user when the user provides an instruction is a state in which the user can make a reasonable decision or judgement. When the user is in no condition to make a reasonable decision or judgement, the processor 24 restricts the instruction from the user. When the user is able to make a reasonable decision or judgement, the processor 24 does not restrict the instruction from the user. The above-described permissible range concerning the difference in biological information is determined in advance. The permissible range may be set for each user. The permissible range may be changed, for example, by a user.

Examples of restricting an instruction are: not accepting an instruction; not allowing a user to input any instruction; receiving an instruction but nullifying it; and receiving an instruction but not executing any of processing, operation, or control in accordance with the instruction.

For example, if an instruction is to make a payment or form a contract, the processor 24 may not accept the instruction, or receive the instruction but nullify it, or receive the instruction but not make a payment or form a contract in accordance with the instruction.

If an instruction is that for the device 14, the processor 24 may not accept the instruction, or receive the instruction but nullify it, or receive the instruction but not operate the device 14 in accordance with the instruction.

If an instruction is that for the service providing apparatus 16, the processor 24 may not accept the instruction, or receive the instruction but nullify it, or receive the instruction but not send information indicating the instruction to the service providing apparatus 16.

Restricting an instruction may be restricting all instructions or restricting a certain instruction but not restricting another instruction.

The processor 24 may restrict an instruction differently in accordance with the state of a user. For example, the processor 24 determines to which degree an instruction is restricted, in accordance with the degree of a state in which a user is unable to make a reasonable decision or judgement. The processor 24 then registers instruction acceptability information indicating to which degree an instruction is restricted in the management information in association with the state information. For example, if a user is in no condition to make a reasonable decision or judgement because of a disease such as epilepsy or a mental disorder, the processor 24 may restrict all instructions from the user. If a user is unable to make a reasonable decision or judgement for a reason other than a disease, the processor 24 may restrict only some instructions from the user. Instructions to be restricted may be determined in advance, or they may be determined by a user.

If multiple items of reference biological information which each deviate from monitored biological information concerning a user by an amount within the permissible range are found, the processor 24 selects the item of reference biological information having the smallest difference from the monitored biological information.

The reference biological information may be information indicating feature components of biological information. In this case, the processor 24 may extract feature components from monitored biological information concerning a user, and search for an item of reference biological information whose feature components are different from those of the monitored biological information by an amount within the permissible range. For example, if brain waves are used as biological information, the processor 24 may extract feature components from monitored brain waves, analyze them, and estimate the state of a user associated with the analyzed brain waves.

To analyze the brain activities (brain waves, for example) of a user, fast Fourier transform (FFT), wavelet transform (WT), time frequency distribution (TFD), eigenvector methods (EM), or auto regressive method (ARM) may be used.

Biological information concerning a user when an instruction is provided from the user may be information monitored at a time when the user has provided an instruction. Biological information concerning a user may alternatively be information monitored at a time when a user has provided an instruction and during a period prior to and subsequent to this time. Biological information concerning a user may alternatively be information monitored during a period prior to a time when a user has provided an instruction or during a period subsequent to this time. Each of the period prior to and subsequent to a time when a user has provided an instruction, the period prior to this time, and the period subsequent to this time is a predetermined period, and may be changed, for example, by a user.

If it is determined that a user is in no condition to make a reasonable decision or judgement, based on biological information concerning the user monitored during a period prior to a time when the user has provided an instruction, the processor 24 restricts this instruction from this user. For example, the processor 24 may not accept the instruction, not allow the user to input the instruction, receive the instruction but nullify it, or receive the instruction but not execute any of processing, operation, or control in accordance with the instruction.

If it is determined that a user is in no condition to make a reasonable decision or judgement, based on biological information concerning the user monitored at a time when the user has provided an instruction or during the period subsequent to this time, the processor 24 may receive the instruction but nullify it, or receive the instruction but not execute any of processing, operation, or control in accordance with the instruction.

The processor 24 is configured to control the operations of the individual elements of the information processing apparatus 10. The processor 24 may include a memory.

Processing executed by the processor 24 may be executed by an apparatus or a device other than the information processing apparatus 10, such as the device 14 or the service providing apparatus 16. Then, information indicating the processing results may be sent to the information processing apparatus 10 and be displayed on the display of the UI 20 or be output as sound information.

For example, the device 14 or the service providing apparatus 16 may restrict an instruction from a user. For example, when an instruction for the device 14 is provided from a user, the processor 24 judges whether the user is capable of making a reasonable decision or judgement, based on biological information concerning the user. The processor 24 then sends information indicating the judging result and information indicating the instruction to the device 14. The device 14 determines whether to restrict the instruction, based on the judging result. If the user is capable of making a reasonable decision or judgement, the device 14 does not restrict the instruction. If the user is in no condition to make a reasonable decision or judgement, the device 14 restricts the instruction. Likewise, when an instruction for the service providing apparatus 16 is provided, the service providing apparatus 16 may restrict the instruction in a manner similar to the device 14.

The processor 24 may send biological information which has not been analyzed to the device 14 or the service providing apparatus 16. Then, the device 14 or the service providing apparatus 16 may judge whether a user is capable of making a reasonable decision or judgement, based on the received biological information, and determine whether to restrict the instruction based on the judging result. In this case, management information is stored in the device 14 or the service providing apparatus 16.

An instruction from a user may be input into a terminal device, such as a PC, a laptop PC, a smartphone, or a cellular phone, used by the user. Then, the information processing apparatus 10, the device 14, the service providing apparatus 16, or another apparatus may judge whether to restrict this instruction. If it is judged that the instruction is not restricted, it is accepted by the terminal device and information indicating this instruction is sent to the device 14, the service providing apparatus 16, or another apparatus directly or via the information processing apparatus 10. If it is judged that the instruction is restricted, any one of the terminal device, the information processing apparatus 10, the device 14, the service providing apparatus 16, and another apparatus may restrict the instruction.

A specific example of the management information will be explained below.

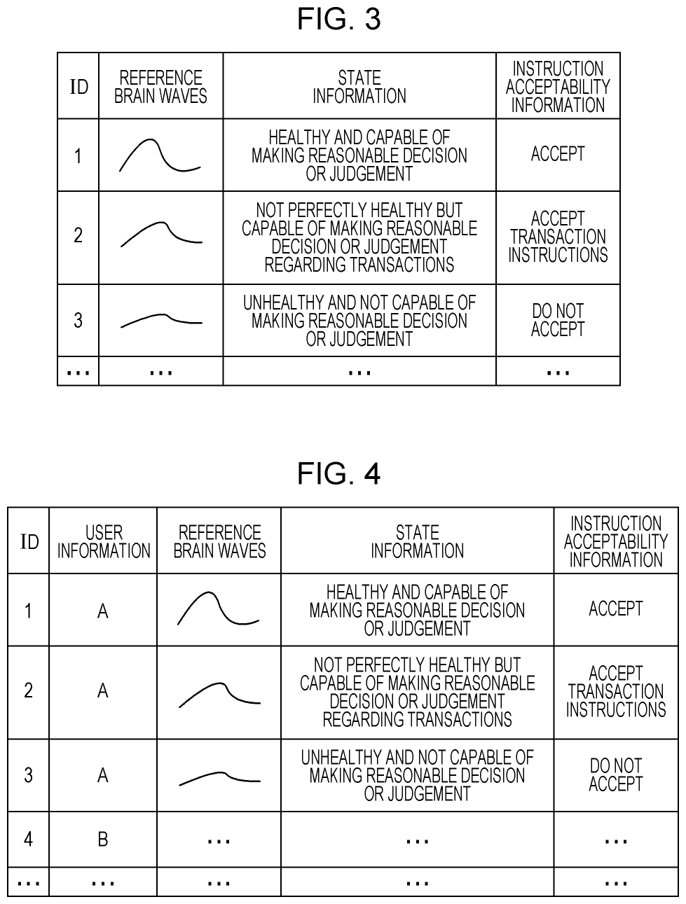

FIG. 3 illustrates an example of a management table, which is an example of the management information. Data in the management table is stored in the storage unit 22. Instead of in the storage unit 22, data in the management table may be stored in another apparatus other than the information processing apparatus 10, such as the device 14, the service providing apparatus 16, or a server.

In the management table, the ID, reference brain waves, state information, and instruction acceptability information are linked with each other.

The ID is information for managing a corresponding item of information registered in the management table.

Reference brain waves are an example of reference biological information. Biological information other than brain waves may be used. The reference brain waves are determined by executing statistical processing, for example. The reference brain waves are brain waves that are assumed to be typically generated from a user being in the state indicated by the state information linked with the reference brain waves. The reference brain waves may be brain waves in a specific frequency band or may include brain waves in multiple frequency bands.

The state information is information indicating whether a user is capable of making a reasonable decision or judgement. The state information may be information indicating whether a user is able to make a reasonable decision or judgement regarding all issues or that regarding specific issues.

The instruction acceptability information is information indicating whether to restrict an instruction from a user.

The state information linked with the reference brain waves of the ID “1” indicates that a user is mentally healthy and is capable of making a reasonable decision or judgement. Hence, the instruction acceptability information linked with the reference brain waves and the state information of the ID “1” indicates that instructions from this user are accepted.

The state information linked with the reference brain waves of the ID “2” indicates that a user is not perfectly mentally healthy but is capable of making a reasonable decision or judgement regarding transactions. Hence, the instruction acceptability information linked with the reference brain waves and the state information of the ID “2” indicates that instructions concerning transactions from this user are accepted, but instructions concerning the other issues are not accepted.

The state information linked with the reference brain waves of the ID “3” indicates that a user is mentally unhealthy and is not capable of making a reasonable decision or judgement. Hence, the instruction acceptability information linked with the reference brain waves and the state information of the ID “3” indicates that instructions from this user are not accepted.

For example, if brain waves concerning a user deviating from the reference brain waves of the ID “1” by an amount within the permissible range are monitored, the processor 24 determines that the user is in a healthy state and does not restrict instructions from the user.

The processor 24 may calculate the degree of similarity between the monitored brain waves concerning a user and the reference brain waves, and judge whether the calculated degree of similarity is higher than or equal to a threshold. The threshold corresponds to a value within the permissible range. If the degree of similarity between the monitored brain waves and the reference brain waves is higher than or equal to the threshold, the processor 24 determines that the monitored brain waves and the reference brain waves are similar to each other. That is, the processor 24 judges that the difference between the monitored brain waves and the reference brain waves is contained within the permissible range. If the degree of similarity between the monitored brain waves and the reference brain waves of the ID “1” is higher than or equal to the threshold, the processor 24 does not restrict instructions from the user.

Instead of the information processing apparatus 10, the device 14 or the service providing apparatus 16 may judge whether to accept instructions from a user, based on the management table shown in FIG. 3, and may restrict instructions based on the judging result.

For each user, the reference biological information, state information, and instruction acceptability information may be linked with each other and be registered in the management table. For example, monitored biological information concerning a user may be registered in the management table as reference biological information concerning this user.

FIG. 4 illustrates an example of the management table in which items of specific reference biological information concerning individual users are registered. In the management table shown in FIG. 4, the ID, user information, reference brain waves, which are an example of the reference biological information, state information, and instruction acceptability information are linked with each other. The user information is information for identifying a user, such as the user name or user ID.

The reference brain waves linked with certain user information are brain waves which concern the user indicated by the user information and which are monitored when this user is in the state indicated by the state information linked with the reference brain waves. For example, for each user and for each state, brain waves concerning a user are monitored in advance and are registered in the management table as the reference brain waves.

For example, when a user A is mentally healthy and is capable of making a reasonable decision or judgement, the brain waves of the user A are monitored. The brain waves are then registered in the management table as the reference brain waves associated with the user information concerning the user A and the state information that “user is mentally healthy and is capable of making a reasonable decision or judgement”. In this case, the instruction acceptability information linked with the user information and the state information indicates that “instructions are accepted”. These items of information may be registered by the information processing apparatus 10 or another apparatus. In the example shown in FIG. 4, these items of information are registered as those of the ID “1”. Individual items of information concerning another user and another state are registered in a similar manner.

Brain waves concerning a user may be monitored multiple times, and then, the average brain waves may be registered as the reference brain waves.

While the user A is logging in the information processing apparatus 10, if brain waves concerning the user A deviating from the reference brain waves of the ID “1” by an amount within the permissible range are monitored, the processor 24 determines that the user A is mentally healthy and is capable of making a reasonable decision or judgement. In this case, the processor 24 does not restrict instructions from the user A.

If brain waves concerning the user A deviating from the reference brain waves of the ID “2” by an amount within the permissible range are monitored, the processor 24 determines that the user A is not perfectly mentally healthy but is capable of making a reasonable decision or judgement regarding transactions. In this case, the processor 24 does not restrict instructions concerning transactions but restricts instructions concerning the other issues.

If brain waves concerning the user A deviating from the reference brain waves of the ID “3” by an amount within the permissible range are monitored, the processor 24 determines that the user A is mentally unhealthy and is not capable of making a reasonable decision or judgement. In this case, the processor 24 restricts instructions from the user A.

Concerning a user other than the user A, individual items of information are registered in the management table in a similar manner.

Processing executed by the information processing system according to the exemplary embodiment will be described below in greater detail.

When it is determined that an instruction from a user is restricted, the processor 24 may output this information. For example, the processor 24 may display this information on the display of the UI 20 or output this information from a speaker as sound information. The processor 24 may send the information to the terminal device used by the user and display it on the terminal device. For example, when the processor 24 has determined that it does not accept an instruction from a user, it outputs this information.

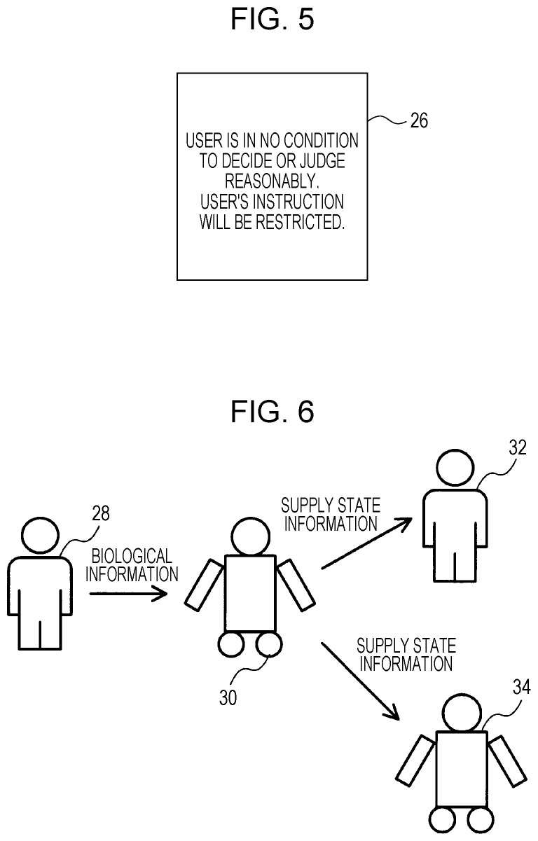

FIG. 5 illustrates an example of information displayed on a screen 26 indicating that an instruction from a user will be restricted. The screen 26 may be a screen displayed on the display of the UI 20 or that on the display of the terminal device of the user.

On the screen 26, a message is displayed. The message indicates that the user is in no condition to make a reasonable decision or judgement and an instruction from the user will be restricted. It can be said that this message is a warning indicating that an instruction will not be accepted. For example, if it is judged that a user is not capable of making a reasonable decision or judgement because of its poor health or its disease, the above-described warning is displayed. The message may be displayed on the screen 26 and/or emitted from a speaker as sound information.

The message shown in FIG. 5 may be displayed when a user has provided an instruction by using the UI 20 or the terminal device of the user, or when the state of the user is estimated based on biological information concerning the user regardless of whether the user has provided an instruction, or in a certain situation where the user provides an instruction. Examples of the situation where the user provides an instruction are situations where the user makes a payment, where the user forms a contract, and any other situations where the user needs to make a decision. More specifically, in a situation where a user purchases a product via web shopping, the message may be displayed before the user gives a purchase instruction or after the user has given a purchase instruction and before it provides a final payment instruction. The message may be displayed when the user has accessed or logged in a shopping site. By taking a shopping site as an example, as a result of restricting an instruction provided from a user, the user can be prevented from purchasing a product or a service that may not be necessary for the user. Additionally, the user can also recognize whether it is capable of making a reasonable decision or judgement regarding the purchasing of a product or a service.

A user may be unable to make a reasonable decision or judgement due to a reason other than its poor health condition or disease. For example, when a user is mentally pressured by hard sell, for example, a message indicating that the user is unable to make a reasonable decision or judgement may be displayed or output as sound.

When it is determined that an instruction from a user is not restricted, the processor 24 may output this information. For example, the processor 24 may display this information on the display of the UI 20 or output it from a speaker as sound information. The processor 24 may send this information to the terminal device of the user and display it on the terminal device.

This will be explained more specifically by taking making a payment or forming a contract as an example. It can be said that a transaction, such as payment or a contract, conducted without restricting an instruction is a transaction conducted when the parties concerned are capable of making a reasonable decision or judgement. If a transaction is conducted when the parties concerned are capable of making a reasonable decision or judgement, it can be assumed that the transaction has successfully been completed under the normal conditions. For example, when both the seller and the purchaser of a product or a service can make a reasonable decision or judgement, a transaction is successfully completed. That is, it can be said that the acceptability of an instruction reflects the certainty of a transaction. In other words, a transaction, such as payment or a contract, conducted without restricting an instruction, is assumed to be a transaction successfully completed under the normal conditions. A judgment whether a purchaser of a product is capable of making a reasonable decision or judgement may also be made when it receives the product. An agreement may be made between a seller and a purchaser that, when the purchaser is in a normal condition when making a payment for a product and also when receiving the product, the purchaser is unable to refuse the product.

When an instruction from a subject user is restricted (when an instruction is not accepted), the processor 24 may supply the state information indicating the state of the subject user to at least one of another user and a device, such as a robot. The device may be the device 14 shown in FIG. 1, a robot assisting the subject user, a user doing some work, or a terminal device used by a user.

FIG. 6 illustrates an example in which the processor 24 supplies such notification. Biological information concerning a user 28 is monitored and is sent to a robot 30. The robot 30 contains the information processing apparatus 10 and judges whether the user 28 is capable of making a reasonable decision or judgement, based on the received biological information, and determines whether to restrict an instruction from the user 28 based on the judging result. If the robot 30 has determined that an instruction from the user 28 is restricted, the processor 24 integrated within the robot 30 supplies the state information indicating the state of the user 28 to a user 32 or a robot 34 located near the robot 30. The user 32 or the robot 34 may be detected by a sensor, for example, provided in the robot 30, or be registered in the robot 30 as a user or a device to receive state information. The processor 24 may output sound indicating the state of the user 28 from a speaker, display an image indicating the state of the user 28 on the display of the UI 20, or send the state information to the terminal device of the user 32 or the robot 34. This enables the user 32 or the robot 34 to recognize that the user 28 is in no condition to make a reasonable decision or judgement.

When an instruction from a subject user is not restricted (when an instruction is accepted), the processor 24 may supply the state information indicating the state of the subject user to at least one of another user and a device, such as a robot. In the example shown in FIG. 6, the processor 24 integrated within the robot 30 supplies the state information indicating the state of the user 28 to the user 32 or the robot 34. The processor 24 may supply the state information in a manner similar to a case in which an instruction is restricted. This enables the user 32 or the robot 34 to recognize that the user 28 is able to make a reasonable decision or judgement. When the user 28 is in a normal condition, the user 32 may go to work and robot 34 may make communications, for example.

The robot 30 may not contain the information processing apparatus 10. In this case, biological information concerning a user is sent to the information processing apparatus 10 via the robot 30, and the information processing apparatus 10 judges whether the user is capable of making a reasonable decision or judgement. Then, information indicating the judging result is sent from the information processing apparatus 10 to the robot 30. The robot 30 then supplies the state information indicating the state of the user to the user 32 or the robot 34 in accordance with the judging result.

The robot 30 may store history information indicating communication with the user 28 and supply it to the user 28. For example, the robot 30 may notify the user 28 of the state of the user 28 or a change in the state of the user 28 or inform the user 28 that a right to restrict an instruction from the user 28 is given to the robot 30.

The processor 24 may restrict an instruction from a user in accordance with a history of the behavior of a user and the state of the user. For example, for each user, behavior history information indicating the behavior history of a user is stored in the information processing apparatus 10 or another apparatus, such as a server. The behavior history information may be stored in the robot 30 shown in FIG. 6. Examples of the behavior history are a product/service purchase history, a dietary history, an exercise history, and a traveling history. The robot 30 may detect the behavior of a user and store information indicating the behavior of the user as behavior history information. Alternatively, a user may store behavior history information in the information processing apparatus 10, the robot 30, or another device. A device other than the robot 30 may detect the behavior of a user.

It is judged, for example, whether a user has a habit of wasting its money, based on the purchase history of the user. This judgement is made by the processor 24 or a device other than the information processing apparatus 10. If it is determined, based on biological information concerning a user having a habit of wasting its money, that this user is in no condition to make a reasonable decision or judgement, the processor 24 restricts some instructions from this user. For example, the processor 24 restricts instructions to purchase a product or a service and do not restrict the other instructions.

It is judged, for example, whether a user has a symptom of bulimia, based on the dietary habits of the user. This judgement is made by the processor 24 or a device other than the information processing apparatus 10. If it is determined, based on biological information concerning a user having a symptom of bulimia, that this user is in no condition to make a reasonable decision or judgement, the processor 24 restricts some instructions from this user. For example, the processor 24 restricts instructions to purchase or order food and drink.

When it is determined that an instruction from a user is restricted, the processor 24 may suspend the execution of the instruction from the user. For example, if it is determined based on biological information concerning a user that the user is in an unhealthy state and is in no condition to make a reasonable decision or judgement, the processor 24 suspends the execution of an instruction from the user. In this case, the processor 24 stores information indicating the content of the instruction from the user in the storage unit 22 or another device. Then, when it is determined that an instruction from the user is no longer restricted, the processor 24 outputs a request to execute the stored instruction to the user. For example, when it is determined based on the biological information concerning the user that the user is in a healthy state and is able to make a reasonable decision or judgement, the processor 24 outputs a request to execute the stored instruction to the user. For example, the processor 24 may display this request on the display of the UI 20, or send this request to the terminal device of the user and display it on the terminal device, or output this information from a speaker as sound information.

This will be explained through a specific example. When a user has provided an instruction to make a payment while being in a poor health condition, the processor 24 suspends the execution of this instruction. Then, when the user has recovered from a poor health condition, the processor 24 outputs a request to make the payment to the user.

When it is determined that an instruction from a subject user is restricted, the processor 24 may output a request to provide an instruction on behalf of the subject user to another user. The processor 24 outputs this request to another user who can provide an instruction on behalf of the subject user. For example, when it is determined based on biological information that a subject user is in an unhealthy state and is in no condition to make a reasonable decision or judgement, the processor 24 outputs a request to provide an instruction on behalf of the subject user to another user. Such a user may be called a substitute user.

A substitute user is a user whose instructions are not restricted. For example, a substitute user is a user whose biological information indicates that it is mentally healthy, that is, the substitute user is capable of making a reasonable decision or judgement. Biological information concerning a substitute user is monitored by the biological information monitoring apparatus 12 and is sent to the information processing apparatus 10. The processor 24 determines the state of the substitute user based on the received biological information. If there are multiple users who are mentally healthy and can make a reasonable decision or judgement, the processor 24 may output a substitute request to the healthiest user.

Examples of outputting a request to provide an instruction on behalf of a subject user are sending a request message to a terminal device used by a substitute user, outputting this message from a speaker as sound information, and displaying this message on the display of the UI 20. Upon receiving this message, the substitute user may provide an instruction on behalf of the subject user. For example, the substitute user may provide an instruction to make a payment for a product or to form a contract or another instruction. The substitute user may provide an instruction on behalf of the subject user when it is received a request from the subject user.

The processor 24 may store biological information monitored when a user has provided an instruction and indicating that the user is in a healthy state. The biological information is stored in the storage unit 22 for each user. That is, the processor 24 may store biological information monitored when a user is in a healthy state in the storage unit 22 or another device. Such biological information concerning a user may be monitored multiple times, and the monitored items of biological information or the average thereof may be stored in the storage unit 22 or another device as biological information indicating that the user is in a healthy state.

Then, for each user, the processor 24 checks biological information monitored when a user has provided an instruction against the stored biological information concerning this user. If the monitored biological information is found to be included in a certain range of similarity with the stored biological information, the processor 24 accepts the instruction without restricting it. For example, if the difference between the monitored biological information and the stored biological information is included within a permissible range, the monitored biological information is found to be included in the certain range of similarity with the stored biological information.

The definition of the healthy state (the normal or regular state, for example) may be different among users. If biological information in a healthy state is stored for each user, a judgement as to whether an instruction from a user is restricted can be made by reflecting the differences in the definition of the healthy state among users. It is also possible to make it difficult to provide an instruction by pretending to be another person.

If biological information concerning a user providing an instruction is not monitored, the processor 24 may restrict this instruction. For example, if biological information concerning a user is not monitored, the processor 24 does not accept any instruction from this user. In this manner, it is possible to prevent a user whose biological information is not monitored from providing an instruction.

In the embodiment above, the term “processor” refers to hardware in a broad sense. Examples of the processor includes general processors (e.g., CPU: Central Processing Unit), dedicated processors (e.g., GPU: Graphics Processing Unit, ASIC: Application Integrated Circuit, FPGA: Field Programmable Gate Array, and programmable logic device). In the embodiment above, the term “processor” is broad enough to encompass one processor or plural processors in collaboration which are located physically apart from each other but may work cooperatively. The order of operations of the processor is not limited to one described in the embodiment above, and may be changed.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a processor configured to
obtain biological information indicating a state of a subject user which is found when an instruction is provided from the subject user, and
determine whether to restrict accepting of the instruction in accordance with the state indicated by the biological information.

2. The information processing apparatus according to claim 1, wherein the processor is further configured to:
output information indicating that the instruction is not accepted if the processor determines that the instruction is not accepted based on the state indicated by the biological information.

3. The information processing apparatus according to claim 1, wherein the processor is further configured to:
supply information indicating the state of the subject user to at least one of a user other than the subject user and a device if the processor determines that the instruction is not accepted based on the state indicated by the biological information.

4. The information processing apparatus according to claim 2, wherein the processor is further configured to:
supply information indicating the state of the subject user to at least one of a user other than the subject user and a device if the processor determines that the instruction is not accepted based on the state indicated by the biological information.

5. The information processing apparatus according to claim 1, wherein the processor is further configured to:
supply information indicating the state of the subject user to at least one of a user other than the subject user and a device if the processor determines that the instruction is accepted based on the state indicated by the biological information.

6. The information processing apparatus according to claim 1, wherein the processor determines whether to restrict accepting of the instruction in accordance with a history of behavior of the subject user and the state indicated by the biological information.

7. The information processing apparatus according to claim 2, wherein the processor determines whether to restrict accepting of the instruction in accordance with a history of behavior of the subject user and the state indicated by the biological information.

8. The information processing apparatus according to claim 3, wherein the processor determines whether to restrict accepting of the instruction in accordance with a history of behavior of the subject user and the state indicated by the biological information.

9. The information processing apparatus according to claim 1, wherein:
the biological information is information indicating a mental state of the subject user; and
the processor is configured to
accept the instruction when the biological information indicates that the subject user is in a healthy state, and
not accept the instruction when the biological information indicates that the subject user is not in a healthy state.

10. The information processing apparatus according to claim 2, wherein:
the biological information is information indicating a mental state of the subject user; and
the processor is configured to
accept the instruction when the biological information indicates that the subject user is in a healthy state, and
not accept the instruction when the biological information indicates that the subject user is not in a healthy state.

11. The information processing apparatus according to claim 3, wherein:
the biological information is information indicating a mental state of the subject user; and
the processor is configured to
accept the instruction when the biological information indicates that the subject user is in a healthy state, and
not accept the instruction when the biological information indicates that the subject user is not in a healthy state.

12. The information processing apparatus according to claim 9, wherein the processor is further configured to:
suspend execution of the instruction if the biological information indicates that the subject user is not in a healthy state, and outputs a request to execute the suspended instruction to the subject user when the biological information indicates that the subject user is in a healthy state.

13. The information processing apparatus according to claim 9, wherein the processor is further configured to:
output a request to provide the instruction on behalf of the subject user to a substitute user if the biological information indicates that the subject user is not in a healthy state, the substitute user being a user who is able to provide the instruction on behalf of the subject user.

14. The information processing apparatus according to claim 13, wherein the substitute user is a user whose biological information indicates that the substitute user is mentally healthy.

15. The information processing apparatus according to claim 9, wherein the processor is further configured to:
for each user, store biological information in a storage, the biological information being information monitored when an instruction is provided from a user and indicating that the user is in a healthy state; and
for each user, accept an instruction if biological information monitored when the instruction is provided from a user is included in a certain range of similarity with the corresponding biological information stored in the storage.

16. The information processing apparatus according to claim 1, wherein the processor is further configured to:
not accept the instruction if biological information concerning the subject user is not monitored.

17. The information processing apparatus according to claim 2, wherein the processor is further configured to:
not accept the instruction if biological information concerning the subject user is not monitored.

18. The information processing apparatus according to claim 3, wherein the processor is further configured to:
not accept the instruction if biological information concerning the subject user is not monitored.

19. The information processing apparatus according to claim 1, wherein the instruction is an instruction to make a payment.

20. A non-transitory computer readable medium storing a program causing a computer to execute a process, the process comprising:
obtaining biological information indicating a state of a subject user which is found when an instruction is provided from the subject user; and
determining whether to restrict accepting of the instruction in accordance with the state indicated by the biological information.
